(19) United States
(12) Patent Application Publication
(10) Pub. No.: US 2010/0062465 A1
(43) Pub. Date: Mar. 11, 2010

(54) Detection of Histone Deacetylase Inhibition

(76) Inventors: Sabrina M. Ronen, Bellaire, TX (US); Juri G. Gelovani, Missouri City, TX (US); William G. Bornmann, Missouri City, TX (US); Jihai Pang, Pearland, TX (US); Ashutosh Pal, Houston, TX (US); William P. Tong, Houston, TX (US); Ashutosh Paz, Houston, TX (US); William P. Tong, Houston, TX (US); David S. Maxwell, Pearland, TX (US); Madhuri Sankaranarayanapillai, Houston, TX (US)

Correspondence Address: Baker Botts L.L.P, 910 Louisiana Street, One Shell Plaza, HOUSTON, TX, 77002, US

(21) Appl. No.: 12/297,832

(22) PCT Filed: Apr. 20, 2007
(86) PCT No.: PCT/US2007/067111

§ 371 Date: Oct. 20, 2008

Related U.S. Application Data
(60) Provisional application No. 60/745,361, filed on Apr. 21, 2006.

(51) Int. Cl. C12Q 1/34; C07C 271/10; C07F 7/22; C07C 233/01

(52) U.S. Cl. 435/18; 560/159; 556/87; 564/158

(57) ABSTRACT

Provided are compositions and methods for intracellular detection of enzyme activity. One example of a composition is a histone deacetylase substrate comprising a compound of the following formula (I): One example of a method is a method for detecting histone deacetylase activity comprising introducing a compound according to formula (1) to a plurality of cells and monitoring the cells with magnetic resonance spectroscopy.

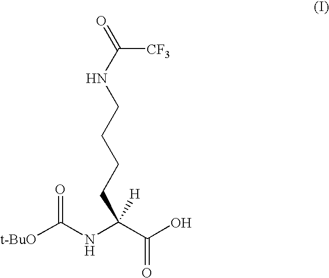

### CROSS-REFERENCE TO RELATED APPLICATIONS

This application claims the benefit of U.S. Provisional Application No. 60/745,361, filed Apr. 21, 2006, which is incorporated herein by reference.

### STATEMENT OF GOVERNMENT INTEREST

This disclosure was developed at least in part using funding from Core Institutional grant to MDACC from NCI (P30 CA016672 30) for the support of Core NMR facility and the Core Analytical facility. The U.S. government may have certain rights in the invention.

### BACKGROUND

Acetylation and deacetylation of nucleosomal core histones play an important role in the modulation of chromatin structure and the regulation of gene expression. The acetylation status of histones is controlled by the activities of histone acetyltransferases (HATs) and histone deacetylases (HDACs) which respectively catalyze removal or addition of acetyl groups onto the ε-amino of lysine residues in the histone tail. Disruptions in HDACs and HATs have been associated with cancer development. Conversely it has been shown that HDAC inhibitors (HDACIs) lead to differentiation, growth arrest, and/or apoptosis in treated cells and tumors. As a result, HDACIs are currently in clinical trials and show promising results in several different tumor types. The exact mechanism of action of HDACIs is not entirely clear. Reactivation of silenced tumor suppressor genes occurs following HDAC inhibition in some cases. However HDACIs can lead not only to gene stimulation but also to gene repression. Nonetheless, many of the modulated genes mediate proliferation, cell cycle progression, or apoptosis, and include p21/WAF1, caspases, p53, vascular endothelial growth factor, Her2/neu, and bcr/abl. In addition, acetylation of nonhistone proteins is likely involved in the activity of HDACIs, and HDAC substrates such as pRb, E2F, and Hsp90.

As mentioned, several HDACIs are currently in clinical trials. However, at present there is no direct noninvasive means to measure drug delivery to the tumor tissue, drug-target interaction, or molecular response. Response to HDACIs in clinical trials is correlated with acetylation of peripheral blood mononuclear cells or acetylation of histones in tumor biopsy specimens. Blood tests are well tolerated by patients, but provide only an indirect indicator of drug delivery and activity at the tumor site. Biopsies reliably assess drug action, but are surgically invasive. A further difficulty is that response in many cases is associated with tumor stasis, rather than shrinkage, limiting the use of traditional imaging methods. Determining the appropriate, biologically relevant, drug dose and assessing drug action at the tumor site, in vivo, present a challenge. A noninvasive method of assessing drug delivery to, and the effect on, the intended molecular target is therefore needed.

Magnetic resonance spectroscopy (MRS) presents a noninvasive nondestructive method, which can provide longitudinal pharmacokinetic and pharmacodynamic biomarkers of drug delivery and action at defined anatomical locations in individual cancer patients. ¹⁹F MRS has been used in studies of fluorinated chemotherapeutic agents in cells, animal models, and patients, and also provides a tool to assess different physiological parameters including oxygenation, pH, and gene expression. In addition, MRS can monitor changes in cellular metabolites that are associated with clinical response to traditional chemotherapy or radiotherapy. An increase in choline containing metabolites, as detected using either ³¹P or ¹H MRS, is associated with cell transformation, and a drop in those metabolites is typically associated with response to treatment. More recently ³¹P MRS has been used to identify biomarkers of response to novel targeted therapies.

### SUMMARY

The present disclosure, according to certain embodiments, is generally directed to compositions and methods for intracellular detection of enzyme activity. More particularly, the present disclosure relates to compounds for assessing inhibition of histone deacetylase activity and associated methods of use. Histone deacetylase (HDAC) inhibitors are new and promising antineoplastic agents. Current methods for monitoring early response rely on invasive biopsies or indirect blood-derived markers. The methods of the present disclosure, according to certain embodiments, provide a magnetic resonance spectroscopy (MRS)-based method to detect HDAC inhibition.

The present disclosure is based in part on the observation that several of the genes and proteins modulated by HDAC inhibition may lead to MRS detectable changes. These include down-regulation of receptor tyrosine kinases and their downstream effector molecules, which have been associated with a drop in phosphocholine (PC); modulation of p53, which could affect PC levels; and Hsp90 acetylation and inhibition, which could lead to increased PC and glycerophosphocholine (GPC). Thus, ³¹P MRS could be used to monitor metabolic changes associated with inhibition of HDAC. However, any metabolic changes observed in the ³¹P spectrum represent indirect and often non-specific downstream events. Specific direct indicators of drug activity on the intended molecular target are therefore needed to complement the downstream metabolic changes.

Accordingly, in certain example embodiments of the present disclosure, a fluorinated HDAC substrate—the lysine derivative Boc-Lys-TFA-OH (BLT)—may be used as a specific spectroscopic indicator to directly monitor HDAC inhibition. In this way, MRS can be used as a method for assessing HDAC inhibition and its downstream signaling and metabolic effects. Such methods may be used clinically to noninvasively monitor drug delivery and/or molecular activity, which could lead to optimized drug scheduling and dosing.

The features and advantages of the present disclosure will be readily apparent to those skilled in the art upon a reading of the description of the embodiments that follows.

### FIGURES

Some specific example embodiments of the disclosure may be understood by referring, in part, to the following description and the accompanying drawings.

FIG. 1 is an illustration of HDAC8 crystal structure with docked BLT into catalytic site. Hydrogen bonds are designated with a line. Red represents oxygen, blue nitrogen, and pink fluorine. The element Zinc is colored gold. The insert represents BLT.

FIG. 2 is a sequential ¹⁹F MRS spectrum of BLT in the presence of recombinant HDAC8 in vitro. Spectra are the result of 128 ¹H-decoupled scans acquired using a 30 degree flip angle and a 3 s relaxation delay. Spectra demonstrate a drop in Boc-Lys-TFA-OH (BLT) as it is cleaved by HDAC8 to form trifluoroacetate (TFA). Insert illustrates the full time course of the experiment.

FIG. 3 are graphs showing the effect on cell proliferation (A) and HDAC activity (B) of HDAC-inhibitor treatment. PC3 cells were treated with 2, 5 and 10 μM para-fluorinated suberoylanilide hydroxamic acid (FSAHA) or with FSAHA in the presence of 1 mM Boc-Lys-TFA-OH (FSAHA+BLT). Cell proliferation was determined using the WST-1 assay and HDAC activity was determined using the Fluor de Lys assay. Error bars represent SD. * P<0.05 compared to DMSO treated controls. n.s. not significant (P>0.05).

FIG. 4A is a representative ¹H-decoupled ¹⁹F MR spectra of PC3 cell extracts. Cells were treated for 24 h with 10 μM p-fluoro-suberoylanilide hydroxamic acid (FSAHA) in the presence of 1 mM Boc-Lys-TFA-OH (BLT) (top) or with 1 mM BLT alone (bottom). Average BLT content in the FSAHA-treated cells was 32 fmol/cell compared to 14 fmol/cell in controls. Spectra are the result of 128 scans acquired using a 30 deg. flip angle and a 3 s relaxation delay. Reference (Ref) was C₆F₆.

FIG. 4B is a graph showing intracellular BLT levels as a function of HDAC inhibition. BLT levels were determined by ¹⁹F MRS as illustrated in A. HDAC inhibition was determined using the Fluor de Lys assay. Error bars represent SD. Line represents the best linear fit. Spearman's rank correlation indicates that BLT levels negatively correlated with HDAC activity (Rho=−0.75; P<0.05).

FIG. 5A is a representative ¹H-decoupled ³¹P MR spectra of PC3 cell extracts. Cells were treated for 24 h with 10 μM p-fluoro-suberoylanilide hydroxamic acid (FSAHA) in the presence of 1 mM Boc-Lys-TFA-OH (top) or with 1 mM BLT alone (bottom). Average phosphocholine (PC) content in the FSAHA-treated cells was 15 fmol/cell compared to 7 fmol/cell in controls. Spectra are the result of 3000 scans acquired using a 30 degree flip angle and a 3 s relaxation delay. Reference (Ref) was MDPA.

FIG. 5B is a graph showing PC levels as a function of HDAC inhibition. PC levels were determined by ³¹P MRS as illustrated in A. HDAC inhibition was determined using the Fluor de Lys assay. Error bars represent SD. Line represents the best linear fit. Spearman's rank correlation indicates that PC levels negatively correlated with HDAC activity (Rho=−0.86; P=0.02).

FIG. 6 is a representative Western blot analysis of c-Raf-1, cdk4, Hsp70 and GAPDH. PC3 were treated with vehicle (DMSO), 1 mM Boc-Lys-TFA-OH (BLT) or 10 μM para-fluorinated suberoylanilide hydroxamic acid (FSAHA) in the presence of 1 mM BLT. GAPDH served as a loading and transfer control.

FIG. 7 is a synthesis (scheme 1) showing synthesis of fluoro-suberanilohydroxamic acids (f-SAHA).

FIG. 8 is a synthesis (scheme 2) of 3-iodo and 3-tributylstannyl suberanilohydroxamic acid.

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are herein described in more detail. It should be understood, however, that the description of specific example embodiments is not intended to limit the disclosure to the particular forms disclosed, but on the contrary, this disclosure is to cover all modifications and equivalents as illustrated, in part, by the appended claims.

### DESCRIPTION

The present disclosure, according to certain embodiments, is generally directed to compositions and methods for intracellular detection of enzyme activity. More particularly, the present disclosure relates to agents for molecular imaging of histone deacetylase activity and and associated methods of use.

The imaging agents of the present disclosure are capable of serving as a substrate based imaging agent for molecular imaging of HDAC activity. Generally, the imaging agents of the present disclosure should not modify the biological effects of HDAC inhibitors. For example, the imaging agents of the present disclosure should not affect HDAC activity or cell proliferation. Furthermore, the imaging agents of the present disclosure should be recognized and cleaved by HDAC to release a detectable cleavage product. Moreover, the imaging agents, prior to cleavage by HDAC, must be detectable. In addition, the intracellular levels of subtrate should be correlated with cellular HDAC activity. Finally, the imaging agents generally should be non-toxic to the cells.

Generally, the imaging agents of the present disclosure may be fluorinated substrates of histone deacytylase. In certain embodiments, imaging agents may comprise compounds represented by the following formula (I):

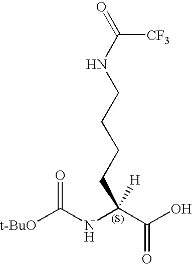

The compound of formula (I) represents fluorinated lysine derivative Boc-Lys-TFA-OH (BLT).

The compounds described herein are intended to include salts, enantiomers, esters, pharmaceutically acceptable salts, hydrates, prodrugs, or solvates thereof, in pure form and as a mixture thereof. Also, when a nitrogen atom appears, it is understood sufficient hydrogen atoms are present to satisfy the valency of the nitrogen atom. The compounds of formula (I) may be synthesized using methods known in the art.

While a chiral structure may be shown above, by substituting into the synthesis schemes an enantiomer other than the one shown, or by substituting into the schemes a mixture of enantiomers, a different isomer or racemic mixture can be achieved. Thus, all such isomers and mixtures are included in the present disclosure. The compounds described may contain asymmetric centers and may thus give rise to diastereomers and optical isomers, the present disclosure is meant to comprehend such possible diastereomers as well as their racemic and resolve, enantiomerically pure forms and pharmaceutically acceptable salts thereof.

The compositions of the present disclosure also may be provided as a pharmaceutical composition comprising a compound of Formula (I) and a pharmaceutically acceptable carrier.

Pharmaceutical compositions may be utilized to administer the compounds of the present disclosure. Such pharmaceutical compositions comprise a compound of Formula I in combination with a pharmaceutically acceptable carrier, and optionally other therapeutic ingredients. The term “salts” refers to salts prepared from pharmaceutically acceptable bases including inorganic bases and organic bases. Representative salts derived from inorganic bases include aluminum, ammonium, calcium, copper, ferric, ferrous, lithium, magnesium, manganic salts, manganous, ammonium, potassium, sodium, zinc, and the like. Particularly preferred are the calcium, magnesium, potassium, and sodium salts. Representative salts derived from pharmaceutically acceptable organic bases include salts of primary, secondary and tertiary amines, substituted amines including naturally occurring substituted amines, cyclic amines, and basic ion exchange resins, such as arginine, betaine, caffeine, choline, NN′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compounds of the present disclosure are basic, salts may be prepared from pharmaceutically acceptable non-toxic acids, including inorganic and organic acids. Examples of such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, and sulfuric and tartaric acids.

In certain embodiments, the imaging agents of the present disclosure may be used to assess HDAC activity. In certain embodiments, the activity of endogenous metabolites affected by HDAC activity may also be monitored in conjunction with assessment of HDAC activity using the imaging agents of the present disclosure. In certain other embodiments, the imaging agents of the present disclosure may be used in conjunction with HDACIs to assess, among other things, HDAC inhibition, drug delivery, drug target interaction, and molecular response. Examples of HDACIs suitable for use in conjuction with the methods and compositions of the present disclosure include, but are not limited to, suberoylanilide hydroxamic acid (SAHA), fluorinated derivatives of suberoylanilide hydroxamic acid (FSAHA), and iodinated and tributylstannanyl derivatives of suberoylanilide hydroxamic acid. In certain embodiments, the HDACIs may comprise a compound represented by the following formula (II):

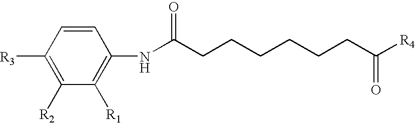

wherein R₁, R₂, and R₃, individualy represent an F or H, and R₄ represents NHOH or OEt. In certain embodiments, R₁ represents F, R₂ represents H, R₃ represents H, and R₄ represents NHOH. In certain other embodiments, R₁ represents H, R₂ represents F, R₃ represents H, and R₄ represents NHOH. In certain other embodiments, R₁ represents H, R₂ represents H, R₃ represents F, and R₄ represents NHOH. In certain other embodiments, R₁ represents F, R₂ represents H, R₃ represents H, and R₄ represents OEt. In certain other embodiments, R₁ represents H, R₂ represents F, R₃ represents H, and R₄ represents OEt. In certain other embodiments, R₁ represents H, R₂ represents H, R₃ represents F, and R₄ represents OEt.

In certain other embodiments, the HDACIs of the present disclosure may comprise a compound represented by the following formula (III):

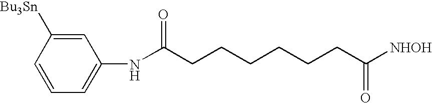

In certain other embodiments, the HDACIs of the present disclosure may comprise a compound represented by the following formula (IV):

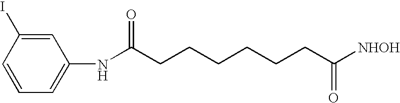

Generally, the imaging agents of the present disclosure may be used to assess HDAC activity using molecular imaging techniques known in the art. In certain embodiments, a cleavage product of HDAC substrate cleavage may be detected using molecular imaging techniques. In certain other embodiments, intact substrate may be detected using molecular imaging techniques. One example of a molecular imaging technique that may be used in conjunction with the methods of the present disclosure, includes, but is not limited to, magnetic resonance spectroscopy (MRS). In certain embodiments, ¹⁹F MRS alone or in combination with ³¹P MRS or ¹H MRS may be used to assess HDAC activity. The use of MRS is advantageous because, among other things, it is a noninvasive method that can be readily translated to the clinical environment.

Detection of a specific cleavage product or an intact substrate using molecular imaging techniques may indicate the effectiveness of the HDACIs, delivery HDACIs to target tissue, HDACIs-target interaction, or molecular response. For example, an imaging agent of the present disclosure may be cleaved in the absence of an HDACI into a detectable cleavage product and a non-detectable cleavage product. In certain embodiments, the imaging agents of the present disclosure may be uncleaved in cells treated with an HDACI, indicating that the HDACI may have inhibited the activity of HDAC. The detection of intact substrate or cleavage product may be used to assess the effectiveness of drug-target interaction. In certain other embodiments, downstream metabolic effects correlated with HDAC inhibition may also be assessed in conjuction with the aforementioned methods. For example, phosphocholine (PC) levels may assessed, which show a negative correlation with HDAC activity.

In certain embodiments, the fluorinated lysine derivative Boc-Lys-TFA-OH (BLT) may be monitored as a ¹⁹F MRS molecular marker of HDAC activity, together with ³¹P MRS of endogenous metabolites. BLT is detectable by ¹⁹F MRS and its cleavage by HDAC may produce TFA and boc-lysine. In silico and in vitro studies confirmed that BLT is a substrate of HDAC8, and therefore may be used as a substrate of other class I and II HDACs. BLT does not affect cell viability or HDAC activity. Importantly, the intracellular levels of BLT, as measured by ¹⁹F MRS, are correlated with cellular HDAC activity. Fluorine in the body is in the form of solid fluorides with very short T₂ relaxation times producing wide and virtually non-detectable MRS peaks. In vivo ¹⁹F MRS therefore presents the advantage that there are no naturally observable fluorinated molecules. Consequently, exogenously administered fluorine-containing compounds are observed without interference. By introducing a fluorinated HDAC substrate it is therefore straightforward to monitor its fate, and thus assess HDAC activity directly in the target tissue.

³¹P MRS provides a noninvasive method for the detection of metabolic biomarkers associated with response to targeted therapies. This methodology may be applied to monitor the downstream metabolic effects correlated with HDAC inhibition, complementing the use of ¹⁹F MRS to monitor drug activity. HDAC inhibition may be accompanied by an increase in PC levels and are PC levels are correlated with the level of this inhibition. This provides a downstream metabolic biomarker of tumor response to HDACI-treatment, further confirming activity of the drug on its target.

In certain other embodiments, the methods and compositions of the present provide a dual method for noninvasively monitoring response to HDACIs. ¹⁹F MRS of the targeted molecular imaging agent BLT can be used to monitor delivery and activity of HDACIs at a tumor site or cancer site, while ³¹P MRS can be used to monitor the downstream metabolic consequences of HDAC inhibition. Together, these two MRS methods provide both a direct marker of HDAC inhibition and a downstream biomarker of cellular response to the inhibition. The combination of both indicators may a more powerful tool than a single marker alone, particularly at lower levels of HDAC inhibition when the changes observed in either marker alone are relatively small. The combination of ¹⁹F and ³¹P (or ¹H) MRS could thus serve as a reliable noninvasive modality to assess HDAC inhibition.

The compositions and methods of the present disclosure may be used therapeutically. In certain embodiments, the compositions of the present disclosure may be administered to a subject using any suitable route of administration for providing a desired dosage of a compound of the present disclosure. In certain embodiments, an intraperitoneal injection may be used. The amount of an imaging agent of the present disclosure that may be administered to a subject may be an amount sufficient to produce an MRS detectable signal without being toxic to the subject. Moreover, the schedule of dosing may be, in certain embodiments, consistent with monitoring the response to HDACIs in vivo. One example of suitable administration means may be an intraperitoneal injection at 100 mg/kg of BLT once a week.

In certain other embodiments, the methods of the present disclosure may be used in detection of HDAC activity in cancer cells, said method comprising administering to a subject amount of an imaging agent of the present disclosure and detecting the imaging agent or its cleavage products using molecular imaging techniques. In certain embodiments, an HDACI may be administered prior to administration of an imaging agent of the present disclosure and inhibition of HDAC activity may be detected non-invasively using molecular imaging techniques.

The following examples are offered by way of illustration and not by way of limitation.

### EXAMPLES

To facilitate a better understanding of the present disclosure, the following examples of specific embodiments are given, which are provided by way of exemplification and not by way of limitation.

### Materials and Methods

In Silico Modeling of BLT Docking Into HDAC.

Docking was performed using the FlexX 1.13.5 software as provided in Sybyl7.1 (Tripos Inc., MI USA) running on a 4-Processor R16000 SGI Tezro. Unless otherwise noted, all defaults were used in the docking experiment. The structure of BLT was drawn into Sybyl using the Sketch module, and types were modified to correspond to the protonated state at pH 7. Charges were not assigned to the molecule, since FlexX uses formal charges that are assigned during the actual run. The crystal structure of HDAC8 complexed with SAHA was retrieved from the RCSB (ID code 1T69) (Berman H M, Westbrook J, Feng Z, et al. The Protein Data Bank. Nucleic Acids Res 2000 January 1;28(1):235-42). The binding site was then defined as 6.5 Å around the SAHA ligand. In the customized setting, the zinc atom was added as a template. For H142 and H143, the histidines were selected to be in the δ protonated state. This particular protonation state was found to be necessary for proper docking of the hydroxamic acid based inhibitors. The CSCORE method (Clark R D, Strizhev A, Leonard J M, Blake J F, Matthew J B. Consensus scoring for ligand/protein interactions. J Mol Graph Model 2002 January;20(4):281-95) was used in the ranking of the 30 requested configurations.

MRS Studies of BLT Cleavage.

To confirm that MRS can be used to monitor cleavage of the HDAC substrate, 0.6 mM BLT (Advance Chem-Tech, KY USA) was incubated alone or with 28U recombinant HDAC-8 (Biomol PA) in a total volume of 500 μl HDAC assay buffer (Biomol PA. USA, composed of 25 mM Tris/Cl, 137 mM NaCl, 2.7 mM KCl, 1 mM MgCl₂ and formulated to maintain HDAC activity). ¹⁹F MRS was used to monitor the decrease in BLT and buildup of the cleavage product trifluoroacetate (TFA) by acquiring ¹H decoupled spectra at 10 min intervals on an Avance DPX 300 Bruker spectrometer (Bruker Biospin, Germany) using a 30 deg. flip angle, 3 s relaxation delay and 128 scans. A sealed insert containing C₆F₆ served as a quantification and chemical shift reference (−164.9 ppm relative to CFCl₃).

Cell Culture, Cell Proliferation and HDAC Activity.

PC3 human prostate cancer cells were routinely cultured in DMEM/F12 (Gibco NY USA) supplemented with 10% FCS (Hyclone, Utah USA) and 10,000 U/mL penicillin 10,000 μg/mL streptomycin and 25 μg/mol amphtenicin B (Gibco, NY USA) at 37° C. in 5% CO₂.

To assess the effect on cell proliferation of BLT the colorimetric WST-1 cell proliferation assay (Roche USA) was used and manufacturer instructions followed. Briefly, 20×10³ cells/well were seeded in 96 well plates. Cells were then treated for 24 hours with BLT at 5 μM, 10 μM, 20 μM, 50 μM, 100 μM, 200 μM, 500 μM, 1 mM, 2 mM, 5 mM and 10 mM or with matched DMSO (1.20,000 to 1.10). Subsequently cells were incubated for 4 hours with the WST-1 cell proliferation reagent and absorbance read at 440 nm using a Tecan Freedom Evo liquid handler equipped with the SAFIRE monochromator-based microplate reader (Tecan U.S., NC, USA).

The effect on cell proliferation of para-fluoro-suberoylanilide hydroxamic acid (FSAHA, synthesized in house based on a previously described method (Stowell J C, Huot R I, Van Voast L. The synthesis of N-hydroxy-N′-phenyloctanediamide and its inhibitory effect on proliferation of AXC rat prostate cancer cells. J Med Chem 1995 April 14;38(8):1411-3), the fluorinated derivative of the clinically relevant HDAC inhibitor suberoylanilide hydroxamic acid (SAHA), was also determined using the WST-1 assay as described above. Cells were treated with 2, 5, and 10 μM FSAHA either in the presence of 1 mM BLT or in the presence of DMSO (in which BLT had to be dissolved first due to its low solubility in growth medium). Note that controls were treated with DMSO in order to clearly identify effects which are due to the compound being investigated rather than its vehicle (DMSO). FSAHA dose was based on previous findings (Butler L M, Agus D B, Scher H I, et al. Suberoylanilide hydroxamic acid, an inhibitor of histone deacetylase, suppresses the growth of prostate cancer cells in vitro and in vivo. Cancer Res 2000 September 15;60(18):5165-70).

The effect on HDAC activity of BLT and the HDAC inhibitors FSAHA and SAHA (also synthesized in house based on the previously described method (Stowell J C, Huot R I, Van Voast L. The synthesis of N-hydroxy-N′-phenyloctanediamide and its inhibitory effect on proliferation of AXC rat prostate cancer cells. J Med Chem 1995 April 14;38(8):1411-3)) was determined using the Fluor de Lys fluorometric assay (Biomol PA. USA) following manufacturer instructions. Briefly 20×10³ cells/well were seeded in 96 well plates and incubated for 24 h with (a) 1 mM BLT (b) FSAHA at 2, 5, 10 μM (c) SAHA at 2, 5, 10 μM (d) FSAHA at 2, 5, 7, 8, 9, 10 μM in the presence of 1 mM BLT (e) FSAHA at 2, 5, 7, 8, 9, 10 μM in the presence of vehicle (DMSO). The Fluor de Lys substrate was then added for 1 h, medium removed, cells rinsed with PBS and incubated for 10 minutes with the Fluor-de-Lys developer, and fluorescence read at 460 nM using the Tecan microplate reader as above. Results were normalized to cell density as determined using the WST-1 assay in the same 96 well plate.

MRS Studies of HDAC Activity.

For MRS studies, PC3 cells were treated for 24 h with FSAHA at 2, 5, 7, 8, 9 and 10 μM in the presence of 1 mM BLT or with 1 mM BLT alone. Approximately 1×10⁷-1.5×10⁷ cells were then extracted using the dual phase extraction method as previously described (Chung Y L, Troy H, Banerji U, et al. Magnetic resonance spectroscopic pharmacodynamic markers of the heat shock protein 90 inhibitor 17-allylamino, 17-demethoxygeldanamycin (17AAG) in human colon cancer models. J Natl Cancer Inst 2003 November 5;95(21):1624-33, Tyagi R K, Azrad A, Degani H, Salomon Y. Simultaneous extraction of cellular lipids and water-soluble metabolites: evaluation by NMR spectroscopy. Magn Reson Med 1996 February;35(2):194-200.47). Briefly, cells were extensively rinsed with ice-cold saline to remove any residual extracellular BLT and medium. Cells were then fixed in 10 ml of ice-cold methanol, scraped off the surface of the culture flask, collected into glass tubes and vortexed. 10 ml of ice-cold chloroform was then added followed by 10 ml of ice-cold de-ionized water. Following phase separation and solvent removal the water-soluble fraction was reconstituted in 250 μl of deuterium oxide (D₂O) and 250 μl of DMSO for ¹⁹F MR measurements. To perform the ³¹P MR measurement 100 μl of EDTA and 50 μl methylene diphosphonic acid (MDPA) in D₂O were added to a final concentration of 10 mM and 0.35 mM respectively. The number of cells extracted was determined by counting a separate flask of cells. ¹⁹F MR spectra of the water-soluble metabolites were recorded as above. Metabolite concentrations were determined by integration and comparison with the area of the external C₆F₆ reference, normalizing to cell number and correcting for saturation effects. Correction for saturation effects was achieved by also acquiring a quantitative inverse gated fully relaxed spectrum on two different samples and calculating the correction factors which need to be applied to the partially relaxed spectra. It was determined that the T₁ relaxation of BLT is 1 s and that of C₆F₆ is 3 s. The fully relaxed spectrum was therefore acquired using a 90 deg. flip angle and a 15 s relaxation delay (5 times the longest T₁). ³¹P MR spectra were recorded on an Avance DRX500 Bruker spectrometer (Bruker Biospin Germany) using a 30 deg. flip angle and 3 s relaxation delay. Metabolite concentrations were determined by integration and comparison with the area of the internal MDPA reference, normalizing to cell number and correcting for saturation effects (correction factors were determined as above by acquiring a fully relaxed quantitative spectrum using a 90 deg. flip angle and a 30 s relaxation delay).

To monitor the fluorinated metabolites, the lipid phase was reconstituted in 500 μl CDCl₃. To monitor the fluorinated metabolites in cellular protein, the protein pellet obtained during cell extraction was dissolved in 1 ml of 0.5 M NaOH and heated to 60° C. for 1 h. Samples were then analyzed by ¹⁹F MRS as above.

Analysis of TFA in Extracellular Medium.

To assess build-up of TFA in medium, samples of extracellular medium were collected and analyzed using gas chromatography-mass spectroscopy (GC-MS). In order to be able to use standard capillary GC-MS, TFA had to be derivatized. This was done using a modification of the procedure of Scott et al. (Scott B F, Mactavish D, Spencer C, Strachan W M J, Muir D C G. Haloacetic acids in Canadian lake waters and precipitation. Environmental Science & Technology 2000;34:4266-72) as follows. One ml of sample, or TFA standard in medium, was treated with 20 mg NaCl, 60 μL HCl, and 30 μl each of 100 mM 1,3-dicyclohexylcarbohiimide (Sigma-Aldrich Chemical Co., MO. USA) and 2,4-difluoroaniline (Sigma-Aldrich Chemical Co., MO. USA) and adjusted to a final volume of 1.320 ml in ethyl acetate. Mixture was vortexed for 60 minutes at room temperature; an additional 50 mg of NaCl added and sample briefly vortexed. Following phase separation the organic layer was removed and stored. The aqueous phase was further extracted twice with 200 μl of ethyl acetate. All three ethyl acetate extracts were combined and treated with 50 μl 3M HCl and 50 μL saturated anhydrous sodium sulfate (Na₂SO₄), vortexed and another 20 mg of Na₂SO₄ added and mixed. The organic phase was then evaporated to dryness under a dry nitrogen gas stream. The residue was dissolved in 250 μL of toluene and transferred into sample vials for analysis. Three microliters of sample extract were analyzed using an Agilent 6890N GC coupled to 5973N MSD in splitless injection mode. TFA was resolved using a Supleco Omegawax 250 capillary column (30 m×0.25 mm). Column temperature was 100° C. for one minute, ramping to 230° C. at 25° C. per minute and then held at 230° C. for two minutes. Detection was performed in EI positive mode monitoring the m/z 225 ion.

Western Blot Analysis of Protein Levels.

PC3 cells were lysed using cell lysis buffer (0.1% NP-40, 50 mM HEPES (pH 7.4), 250 mM NaCl, 1 mM dithiothreitol, 1 mM EDTA, 1 mM NaF, 10 mM β-glycerophosphate, 0.1 mM sodium orthovanadate, 1 μl/ml of protease inhibitor cocktail set III (Calbiochem, USA) and 1 mM phenylmethylsulfonyl fluoride). Lysates were centrifuged at 12,000 rpm for 10 min (4° C.), the protein supernatant collected and total protein concentrations determined using Biorad DC Protein assay reagents (Biorad, CA, USA). Proteins were separated by sodium do-decyl sulfate-polyacrylamide gel electrophoresis using 10% gels and transferred electrophoretically to 0.45 μm Nitrocellulose membranes. Membranes were blocked in blocking buffer containing 5% non-fat dry milk in Tris buffered saline (pH 7.6) and 0.1% Tween-20 and incubated overnight at 4° C. with primary antibodies as follows. c-Raf, 1:1000, (Cell Signaling Technology (CST), MA, USA), Cdk4, 1:2000 (CST, MA, USA), Hsp70, 1:2000 (Stressgen, Canada) and GAPDH, 1:5000 (Stressgen, Canada). This was followed by 1 hour incubation with horseradish peroxidase-conjugated secondary anti-rabbit (CST MA, USA) and anti-mouse (CST, MA, USA) antibodies at dilutions of 1:1000 and 1:2000 respectively. Membranes were washed with enhanced chemiluminescence reagents (LumiGLO & Peroxide, CST MA. USA) for 1 minute and exposed to hyperfilm (Amersham Biosciences, USA), which was developed on a Konica SRX-101 automatic developer (Konica, Tokyo, Japan).

Statistical Analysis.

All results represent the average of at least 3 experiments and are expressed as mean±SD. Data were analyzed by performing a Wilcoxon-Mann-Witney Rank Test and a P value<0.05 was considered significant. A Spearman's rank correlation was used to analyze correlations. KaleidaGraph (Synergy Software, VT USA) and Statistica (Statsoft, OK USA) software were used.

Chemical Synthesis.

All chemicals and solvents were obtained from Sigma-Aldrich (Milwaukee, Wis.) of Fisher Scientific (Pittsburg, Pa.) and used without further purification. ¹H-NMR and ¹³C-NMR spectra were recorded on an IBM-Brucker Avance 300 (300 MHz for ¹H-NMR and 75.48 MHz for ¹³C-NMR), and IBM-Brucker Avance 500 (500 MHz for ¹H-NMR and 125.76 MHz for ¹³C-NMR), spectrometers. Chemical shifts (δ) are determined relative to CDCl₃ (referenced to 7.27 ppm (δ) for ¹H-NMR and 77.0 ppm for ¹³C-NMR) or DMSO-d6 (referenced to 2.49 ppm (δ) for ¹H-NMR and 39.5 ppm for ¹³C-NMR). Proton-proton coupling constants (J) are given in Hertz and spectral splitting patterns are designated as singlet (s), doublet (d), triplet (t), quadruplet (q), multiplet or overlapped (m), and broad (br). Low resolution mass spectra (ionspray, a variation of electrospray) were acquired on a Perkin-Elmer Sciex API 100 spectrometer or Applied Biosystems Q-trap 2000 LC-MS-MS. Flash chromatography was performed using Merk silica gel 60 (mesh size 230-400 ASTM) or using an Isco (Lincon, NE) combiFlash Companion or SQ16x flash chromatography system with RediSep columns (normal phase silica gel (mesh size 230-400ASTM) and Fisher Optima TM grade solvents. Thin-layer chromatography (TLC) was performed on E.Merk (Darmstadt, Germany) silica gel F-254 aluminum-backed plates with visualization under UV (254 nm) and by staining with potassium permanganate or ceric ammonium molybdate.

Synthesis of HDACIs

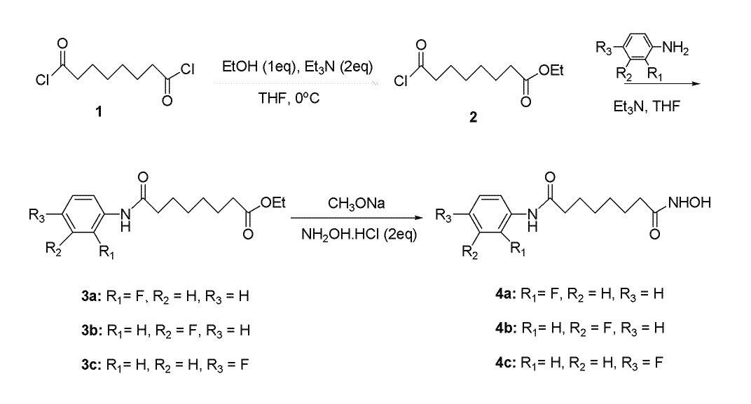

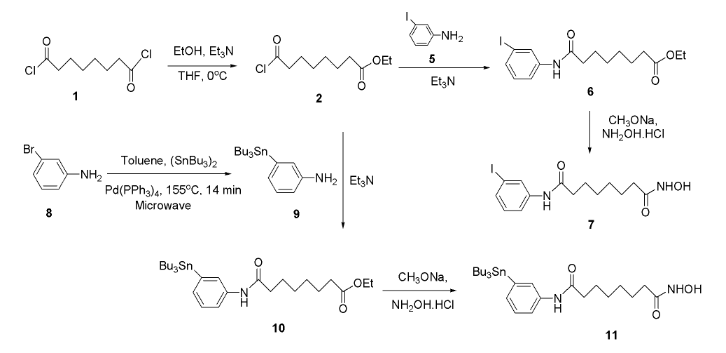

A series of structurally simple fluoro, iodo and tributylstannane suberanilohydroxamic acid have been synthesized. Subaryl chloride was served as the starting material (Stowell J., Huot R., and VanVoast L., J. Med. Chem. 38: 1411 (1995)). The mono amide monoesters (compounds 3 in FIG. 7) were synthesized by reacting subaryl chloride with one equivalent of alcohol followed by one equivalent of fluoro-anilins. The compounds 3 when treated with methanolic hydroxylamine hydrochloride and sodium methoxide yielded f-SAHA 4 in 90-94% yield. For the synthesis of compounds 7 and 11 (3-iodo suberanilohydroxamic acid and 3-butylstannyl suberanilohydroxamic acid) shown in FIG. 8, were used same methodology like compounds 4 except starting material 3-aminophenyltributylstannane (9) was prepared from 3-bromoaniline (8) by microwave technology (Khawli L. A., Kassis A. I.; Nucl. Med. Biol. 19: 297 (1992)).

The final product should be manipulated with a glass (not metal) spatula. If the compound contacts metal when wet, an orange stain occurs.

Octanoic acid, 8-oxo-8-(2′-fluorophenyl), ethyl ester (3a of FIG. 7). To a three-necked 500 mL round-bottomed flask was added 6 mL (7.03 g, 33.1 mmol) of suberoyl chloride and 40 mL of dry THF, and the solution was chilled to 0° C. Through an addition funnel was added dropwise over a 3 hours period a solution of 40 mL THF, 1.9 mL (1.52 g, 33.1 mmol) EtOH and 4.63 mL (3.37 g, 33.1 mmol) triethylamine. Upon completion of the addition, a solution of 60 mL THF, 4.6 mL triethylamine, and 3.78 g of 2-fluoroaniline (33.25 mmol) was added dropwise, and the solution was stirred overnight at room temperature.

To the solution containing white precipitate was added 50 mL of distilled water, and after concentrating, the solution was transferred to a separatory funnel with 20 mL of 1 M NaOH and 100 mL CH₂Cl₂. Layers were shaken and separated. The organic layer was washed twice with 40 mL water, and was evaporated in vacuo and dried to afford 3.5 g of white solid with characteristic fragrant smell. The crude product was purified by flash column chromatography over silica gel, using polarity gradient 20-30% EtOAc in hexane to yield ester 3a (3.05 g 31%) as a white solid: ¹H NMR (CDCl₃) δ 7.52 (t, J=7.8 Hz, 1H), 7.64 (s, 1H), 7.04 (m, 3H), 4.12 (dd, J=7.2 and 0.6 Hz, 2H), 2.38 (t, J=7.2 Hz, 2H), 2.26 (m, 2H) 1.72 (quin, J=6.9 Hz, 2H), 1.61 (quin, J=6.9 Hz, 2H), 1.37 (m, 4H), 1.25 (t, J=7.2 Hz, 3H); ¹³C NMR δ 173.6, 171.4, 152.5 (d, J=241 Hz), 126.4 (d, J=10.5 Hz), 124.4 (d, J=4.5 Hz), 124.1 (d, J 7.5 Hz), 122.1, 114.7 (d, J=19.5 Hz), 60.1, 37.4, 34.6, 28.6(2C), 25.2, 24.1, 14.2; MS (C₁₆H₂₂FNO₃) estimated 295.34 found 296.3 (M+H).

Octanoic acid, 8-oxo-8-(3′-fluorophenyl), ethyl ester (3b of FIG. 7). ¹H NMR (CDCl₃) δ 8.12 (s, 1H), 7.50 (d, J=10.8 Hz, 1H), 7.21 (m, 2H), 6.77 (t, J=8.4 Hz, 1H), 4.12 (d, J=7.2 Hz, 2H), 2.34 (t, J=7.2 Hz, 2H), 2.28 (m, 2H) 1.70 (quin, J=6.9 Hz, 2H), 1.61 (quin, J=6.9 Hz, 2H), 1.37 (m, 4H), 1.25 (t, J=7.2 Hz, 3H); ¹³C NMR δ 173.9, 171.8, 162.7 and 162.1 (d, J=243 Hz), 139.8 (d, J 10.5 Hz), 129.9 (d, J 9 Hz), 115.1, 110.7 (d, J=21 Hz), 107.3 (d, J=25.5 Hz), 60.2, 37.4, 34.2, 28.6 (2C), 25.2, 24.6, 14.2; MS (C₁₆H₂₂FNO₃) estimated 295.34 found 296.4 (M+H).

Octanoic acid, 8-oxo-8-(4′-fluorophenyl), ethyl ester (3c of FIG. 7). ¹H NMR (CDCl₃) δ 7.46 (m, 2H), 7.47 (s, 1H), 7.00 (t, J=8.5 Hz, 2H), 4.12 (d, J=7.2 Hz, 2H), 2.31 (m, 4H) 1.72 (quin, J=6.9 Hz, 2H), 1.61 (quin, J=6.9 Hz, 2H), 1.38 (m, 4H), 1.25 (t, J=7.2 Hz, 3H); ¹³C NMR δ 171.8, 169.5, 159.1 and 157.5 (d, J=239 Hz), 138.3 (d, J=10.5 Hz), 121.2 (2C, d, J=7.5 Hz), 115.6 (2C, d, J=7.5 Hz), 60.6, 37.4, 34.5, 28.9 (2C), 25.2, 14.6; MS (C₁₆H₂₂FNO₃) estimated 295.34 found 296.3 (M+H).

2-Fluorosuberanilohydroxamic acid (4a of FIG. 7). To a solution of hydroxylamine hydrochloride (1.45 g, 21 mmol) in MeOH (27 mL), 1 mg of phenolphthalein and then NaOMe (1.72 g, 31.9 mmol) was added. This mixture was stirred for 30 min at room temperature. When sodium chloride precipitated, compound 3a (2.5 g, 8.4 mmol) was added. The reaction mixture was stirred for an additional 16 h at room temperature and then quenched with 43 mL H₂O and glacial acetic acid (3.5 mL). Stirring was continued for 1 h and the resulting precipitate was filtered, and rinsed with water. The solid was dried at room temperature to yield 4a (2.2 g, 92%) as a white solid showing no impurities by thin layer chromatography or ¹H NMR (DMSO-d₆) δ 10.34 (s, 1H), 9.6 (s, 1H), 8.69 (s, 1H), 7.83 (m, 1H), 7.23 (m, 1H), 7.16 (m, 2H), 2.36 (t, J=7.6 Hz, 2H), 1.94 (t, J=7.4 Hz, 2H), 1.56 (quin, J=6.7 Hz, 2H), 1.51 (quin, J=6.2 Hz, 2H), 1.28 (m, 4H); ¹³C NMR δ 172.1, 169.6, 155.1 and 153.4 (d, J=19.5 Hz), 127.7 (d, J=27 Hz), 125.6 (d, J=7.5 Hz), 124.9, 126.6 (d, J 3 Hz), 115.9 (d, J=19.5 Hz), 36.2, 32.7, 28.8 (2C), 25.5 (2C); ¹⁹F NMR (CDCl₃) δ-112.17; MS (C14H19FN2O3) estimated 282.31 found 283.5 (M+H).

3-Fluorosuberanilohydroxamic acid (4b of FIG. 7). ¹H NMR (DMSO-d₆) δ10.35 (s, 1H), 10.08 (s, 1H), 8.68 (s, 1H), 7.61 (dd, J=11.4 and 1.8 Hz, 1H), 7.31 (m, 2H), 6.84 (t, J=8.4 Hz, 1H), 2.30 (t, J=7.2 Hz, 2H), 1.94 (t, J=7.2 Hz, 2H) 1.57 (quin, J=6.6 Hz, 2H), 1.49 (quin, J=6.6 Hz, 2H), 1.27 (m, 4H); ¹³C NMR (DMSO-d₆) δ 172.1, 169.6, 163.4 and 161.8 (d, J=240 Hz), 141.5 (d, J=10.5 Hz), 130.7 (d, J=9 Hz), 115.1, 109.8 (d, J=21 Hz), 106.2 (d, J=25.5 Hz), 36.8, 32.7, 28.8 (2C), 25.4 (2C); ¹⁹F NMR (CDCl3) δ-112.17; MS (C₁₄H₁₉FN₂O₃) estimated 282.31 found 283.4 (M+H).

4-Fluorosuberanilohydroxamic acid (4c of FIG. 7). ¹H NMR (DMSO-d₆) δ10.33 (s, 1H), 9.90 (s, 1H), 7.60 (m, 2H), 7.11 (t, J=8.5 Hz, 2H), 2.27 (t, J=7.3 Hz, 2H), 1.94 (t, J=7.3 Hz, 2H), 1.51 (m, 4H), 1.28 (m, 4H); ¹³C NMR (DMSO-d₆) δ 171.5, 169.6, 159.1 and 157.5 (d, J=239 Hz), 136.2 (d, J=10.5 Hz), 121.2 (2C, d, J=7.5 Hz), 115.6 (2C, d, J=75 Hz), 367, 32.7, 28.8 (2C), 25.4 (2C); ¹⁹F NMR (CDCl₃) δ-118.71 MS (C₁₄H₁₉FN₂O₃) estimated 282.31 found 283.3 (M+H).

3-iodoanilide of monoethyl suberate (6 of FIG. 8). To a three-necked 500 mL round-bottomed flask was added 6 mL (7.03 g, 33.1 mmol) of suberoyl chloride and 40 mL of dry THF, and the solution was chilled to 0° C. Through an addition funnel was added dropwise over a 3 hours period a solution of 40 mL THF, 1.9 mL (1.52 g, 33.1 mmol) EtOH and 4.63 mL (3.37 g, 33.1 mmol) triethylamine. Upon completion of the addition, a solution of 60 mL THF, 4.6 mL triethylamine, and 7.3 g of 3-iodoaniline (33.32 mmol) was added dropwise, and the solution was stirred overnight at room temperature.

To the solution containing white precipitate was added 50 mL of distilled water, and after concentrating, the solution was transferred to a separatory funnel with 20 mL of 1 M NaOH and 100 mL chloroform. Layers were shaken and separated. The organic layer was washed twice with 40 mL water, and was evaporated in vacuo and dried to afford 8.2 g of white solid with characteristic fragrant smell. The crude product was purified by flash column chromatography over silica gel, using polarity gradient 30-50% EtOAc in hexane to yield ester 6 (6.05 g 45%) as a white solid: ¹H NMR (CDCl₃) δ 9.95 (s, 1H), 8.11 (t, J=1.8 Hz, 1H), 7.53 (dd, J=0.9, 2.1 Hz, 1H), 7.50 (dd, J=0.9, 2.1 Hz, 1H), 7.08 (t, J=8.1 Hz, 1H), 4.03 (q, J=6.9 Hz, 2H), 2.27 (p, J=7.3 Hz, 4H), 1.54 (p, J=6.9 Hz, 4H), 1.27 (m, 4H), 1.17 (t, J=7.2 Hz, 3H); ¹³C NMR δ 173.3, 171.9, 141.2, 131.9, 131.2, 127.6, 118.6, 94.9, 60.1, 36.8, 33.9, 28.7, 28.6, 25.3, 24.8, 14.6; MS (C₁₆H₂₂INO₃) estimated 403.0644 found 404.4 (M+H).

N-Hydroxy-N′-[3-I]phenyloctanediamide (7 of FIG. 8). To a solution of hydroxylamine hydrochloride (1.38 g, 20 mmol) in MeOH (25 mL), 1 mg of phenolphthalein and then NaOMe (1.62 g, 30 mmol) was added. This mixture was stirred for 30 min at room temperature. When sodium chloride precipitated, compound 6 (4.03 g, 10 mmol) was added. The reaction mixture was stirred for an additional 16 h at room temperature and then quenched with 50 mL H₂O and glacial acetic acid (4 mL). Stirring was continued for 1 h and the resulting precipitate was filtered, and rinsed with water. The solid was dried at room temperature to yield 7 (3.63 g, 93%) as a white solid showing no impurities by thin layer chromatography or ¹H NMR (DMSO-d₆) δ 10.37 (s, 1H), 9.98 (s, 1H), 8.1 (s, 1H), 7.51 (d, J=8.1 Hz, 1H), 7.36 (d, J=7.8 Hz, 1H), 7.07 (t, J=7.8 Hz, 1H), 2.28 (t, J=7.5 Hz, 2H), 1.94 (t, J=7.2 Hz, 2H), 1.51 (m, 4H), 1.25 (m, 4H); ¹³C NMR δ 172.1, 169.7, 141.2, 131.9, 131.2, 127.7, 118.7, 94.9 36.8, 32.7, 28.8 (2C), 25.5, 25.4; IR 3314, 32.74, 1660, 1620, 1600, 1530, 1442 cm⁻¹; MS (C₁₄H₁₉IN₂O₃) estimated 390.044 found 391.4 (M+H).

m-Aminophenyltributylstannane (9 of FIG. 8). In a microwave tube containing 0.63 g (3.65 mmol) of m-bromoaniline was placed 50.0 mg (0.0443 mmol) of Pd(PPh₃)₄ the tube was then sealed and flushed with argon. To the tube was then added 3 mL of tolune and 1.5 mL (1.71 g, 2.93 mmol) of (SnBu₃)₂. The tube was then placed in the microwave and heated to 155° C. for 14 min. The resulting black mixture was filtered through celite, and the filtrate obtained was evaporated to dryness under reduced pressure. The residue obtained was dissolved in hexane and the solution was applied to a flash column chromatography eluting with hexane/EtOAc (98:2). Rf=0.33 to yield the pure m-aminophenyltributylstannane 9 in 82% yield. ¹H NMR (CDCl₃): δ 7.11 (t, J=7.5 Hz, 1H), 6.84 (d, J=J=7.0 Hz, 1H), 6.79 (d, J=2.5 Hz, 1H), 6.61 (m, 1H), 3.56 (s, 2H), 1.53 (m, 6H), 1.32 (m, 6H), 1.03 (m, 6H), 0.88 (t, J=7.3 Hz, 9H); ¹³C NMR (CDCl₃) δ 146.1, 143.3, 129.0, 127.1, 123.4, 115.4, 29.5(3C), 27.8(3C), 14.1(3C), 9.9(3C); MS (C₁₈H₃₃NSn) estimated 383.1635 Found 384.3 (M+H).

3-tributylstannylanilide of monoethyl suberate (10 of FIG. 8). To a three-necked 500 mL round-bottomed flask was added 6 mL (7.03 g, 33.1 mmol) of suberoyl chloride and 40 mL of dry THF, and the solution was chilled to 0° C. Through an addition funnel was added dropwise over a 3 hours period a solution of 40 mL THF, 1.9 mL (1.52 g, 33.1 mmol) EtOH and 4.63 mL (3.37 g, 33.1 mmol) triethylamine. Upon completion of the addition, a solution of 60 mL THF, 4.6 mL triethylamine, and 12.8 g of aminophenyltributylstannane (33.32 mmol) was added dropwise, and the solution was stirred overnight at room temperature.

To the solution containing white precipitate was added 50 mL of distilled water, and after concentrating, the solution was transferred to a separatory funnel with 20 mL of 1 M NaOH and 100 mL chloroform. Layers were shaken and separated. The organic layer was washed twice with 40 mL water, and was evaporated in vacuo. The crude product was then purified by flash column chromatography over silica gel, using polarity gradient 25-40% EtOAc in hexane to yield the pure ester 10 (9.07 g, 48%) as a liquid: ¹H NMR (DMSO-d₆) δ 9.74 (s, 1H), 7.61 (m, 2H), 7.22 (t, J=7.5 Hz 1H), 7.04 (d, J=6.9 Hz, 1H), 4.03 (dq, J=6.9, 1.8 Hz, 2H), 2.25 (m, 4H), 1.55 (m, 10 H), 1.30 (m, 10H), 1.06 (m, 3H), 0.99 (m, 6H), 0.83 (m, 9H); ¹³C NMR (DMSO-d₆) 173.2, 171.5, 141.7, 139.5, 131.0, 128.5, 126.9, 119.3, 60.0, 36.8, 33.8, 29.0 (3C), 28.8, 28.7, 27.1 (3C), 25.4, 24.8, 24.7, ¹4.5, 13.9 (3C), 9.5 (3C); MS (C₂₈H₄₉NO₃Sn) estimated 567.2734 found 568.5 (M+H).

3-tributylstannyl suberanilohydroxamic acid. (11 of FIG. 8). To a solution of hydroxylamine hydrochloride (1.73 g, 25 mmol) in MeOH (32 mL), 1 mg of phenolphthalein and then NaOMe (2.1 g, 37.5 mmol) was added. This mixture was stirred for 30 min at room temperature. When sodium chloride precipitated, compound 10 (7.08 g, 12.5 mmol) was added. The reaction mixture was stirred for an additional 16 h at room temperature and then quenched with 70 mL H₂O and glacial acetic acid (5 mL). Stirring was continued for 1 h and the resulting gummy yellow product was collected, and the residue was diluted with ethyl acetate and then was washed with water. After it was dried and concentrated, the crude product was purified by flash chromatography (2-10% methanol in dichloromethane) to give 11 (6.2 g, 90%) as a light yellow gummy product: ¹H NMR (DMSO-d₆) δ10.35 (s, 1H), 9.78 (s, 1H), 7.60 (m, 2H), 7.22 (t, J=7.2 Hz, 1H), 7.04 (d, J=7.2 Hz, 1H), 2.28 (t, J=7.2 Hz, 2H), 1.94 (t, J=7.2 Hz, 2H) 1.53 (m, 10H), 1.29 (m, 10H), 1.01 (m, 6H), 1.01 (m, 9H), ¹³C NMR (DMSO-d₆) 173.2, 170.9, 141.8, 138.2, 131.7, 127.8, 127.4, 119.7, 36.6, 32.3, 29.0 (3C), 28.9, 28.6, 27.0 (3C), 25.4, 25.2, 12.7 (3C), 9.0 (3C); MS (C₂₆H₄₆N₂O₃Sn) estimated 554.253 found 554.8 (M+H).

### Results

BLT is a HDAC Substrate.

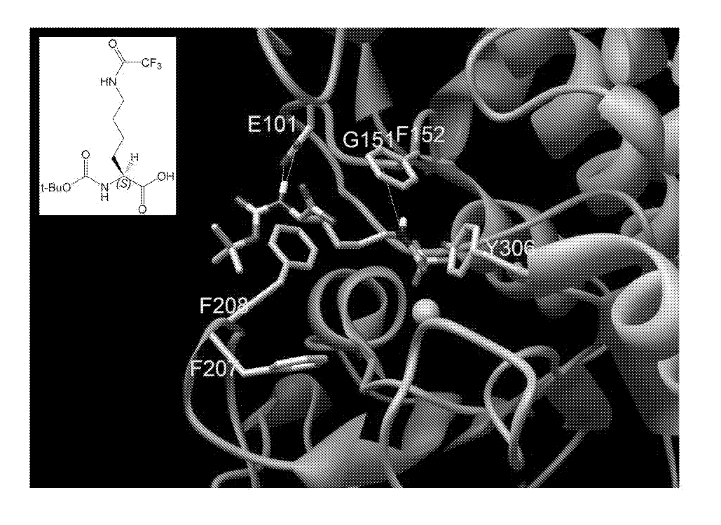

First it was necessary to test the hypothesis that a fluorinated compound composed of a modified lysine could serve as an MRS-detectable substrate of HDAC and could therefore be used to assess HDAC inhibition. The commercially available BLT was investigated and computer modeling was first performed to estimate the BLT-HDAC interaction. The known structure of HDAC8 was used (Berman H M, Westbrook J, Feng Z, et al. The Protein Data Bank. Nucleic Acids Res 2000 January 1;28(1):235-42.). The top-ranking consensus scored configuration of BLT docked with HDAC8 is shown in FIG. 1. The interaction of the carbonyl group with the Y306 is consistent with the proposed model of how acetylated lysine would interact in the catalytic site (Somoza J R, Skene R J, Katz B A, et al. Structural snapshots of human HDAC8 provide insights into the class I histone deacetylases. Structure (Camb) 2004 July;12(7):1325-34.). In the docked structure, there is a hydrogen bond to the backbone carbonyl of G151 and a hydrogen bond between side-chain NH and D101. The aliphatic side-chain interacts with two phenylalanine groups, F152 and F208, and that interaction is also seen in the complex of the SAHA (Bernman et al.). The Boc group consistently selected to position nearby F207 and F208 rather than the area occupied in the reported complex with SAHA. This may result from the asymmetry that is present within the BLT and not within SAHA, which forces the choice between optimizing the orientation of the carboxyl group and the Boc group.

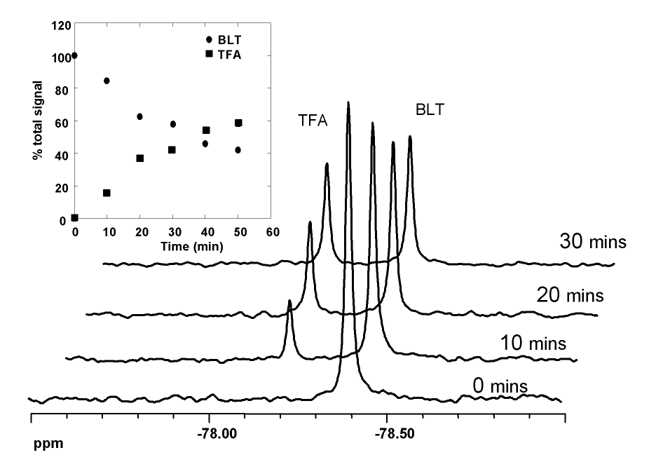

Next ¹⁹F MRS was used to confirm that recombinant HDAC8 does indeed cleave BLT in vitro. As illustrated in FIG. 2, a drop in BLT levels was detected over time accompanied by an increase in TFA levels, consistent with cleavage of BLT by HDAC8 to form TFA and the ¹⁹F MRS invisible boc-lysine. No changes in BLT levels or any buildup of

TFA could be detected in the control sample, which contained no HDAC8. This confirmed that BLT is indeed a substrate of HDAC8 and that its cleavage by HDAC can be monitored by MRS.

BLT Does Not Affect Cell Viability or HDAC Activity.

Before using BLT as a marker of HDAC activity in cells it was necessary to rule out its toxicity. The WST-1 assay was used to investigate the effect on PC3 cells of a range of BLT concentrations from 5 μM to 10 mM compared to matched DMSO controls. BLT did not significantly affect cell proliferation compared to controls up to a concentration of 10 mM (P<0.03 for 10 mM and P>0.4 for all other concentrations from 5 μM to 5 mM, data not shown). We therefore chose to perform MRS experiments with a BLT concentration of 1 mM, which was expected to lead to an MRS detectable signal. Cell numbers following 24-hour treatment with 1 mM BLT represented 95±4% of controls (P>0.1).

Next it was necessary to confirm that 1 mM BLT did not affect HDAC activity in cells. Using the Fluor de Lys assay, we determined that incubation of PC3 cells for 24 hours with 1 mM BLT resulted in HDAC activity levels of 102±9% (P>0.3) relative to DMSO-treated controls indicating no statistically significant effect of 1 mM BLT on HDAC activity.

Inhibition of HDAC Activity and Cell Proliferation by HDACI Treatment is Not Affected by the Addition of BLT.

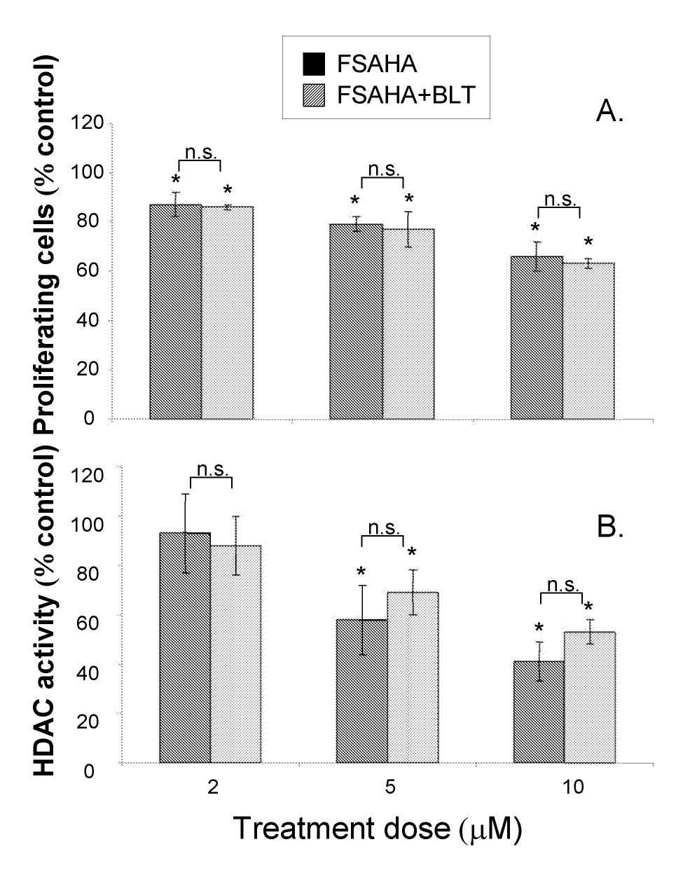

Prior to using MRS of BLT to assess the effect of HDACIs, it was necessary to confirm that the addition of BLT will not modify the biological effects of the HDACI. To this end, both cell proliferation and HDAC activity were investigated. FIG. 3A illustrates the results of these investigations. Treatment with FSAHA, the fluorinated derivative of the HDACI SAHA, resulted in a significant drop in cell proliferation relative to control at all three doses investigated down to 87±5% at 2 μM; 79±3% at 5 μM and 66±6% at 10 μM (P<0.03 for all three doses). Importantly, the presence of BLT did not further affect cell proliferation. In the presence of BLT cell proliferation dropped to 86±1% at 2 μM, 77±7% at 5 μM, and 63±2% at 10 μM (P<0.03 relative to controls and P>0.5 relative to FSAHA). FIG. 3B illustrates the effect on HDAC activity of FSAHA and FSAHA in the presence of 1 mM BLT. 2 μM FSAHA did not lead to a statistically significant drop in HDAC activity (93±16%) but the higher concentrations of FSAHA resulted in significant inhibition of HDAC activity (58±14% at 5 μM and 41±8% at 10 μM, P<0.03). Again, the presence of BLT did not significantly alter the effect of FSAHA treatment (down to 88±12%, 69±9% and 53±5% for 2, 5 and 10 μM respectively P>0.1 relative to FSAHA).

Since SAHA is being investigated in clinical trials, it was necessary to confirm that the biological effects of its fluorinated derivative FSAHA were comparable to those of SAHA. The effects of SAHA and FSAHA on HDAC activity were compared at 2, 5 and 10 μM. The effect of SAHA on HDAC activity was comparable to that observed with FSAHA at 2 and 5 μM (84±10% at 2 μM and 51±3% at 5 μM; P>0.7 relative to FSAHA). However treatment with 10 μM SAHA resulted in a greater inhibition of HDAC activity compared to 10 μM FSAHA (29±3% P<0.03 relative to FSAHA). Nonetheless, it was reasoned that if MRS can detect the effect of FSAHA on HDAC activity, it would also be able to detect the potentially larger effect of SAHA.

¹⁹F MRS of BLT Can be Used to Assess HDAC Inhibition in Cells.

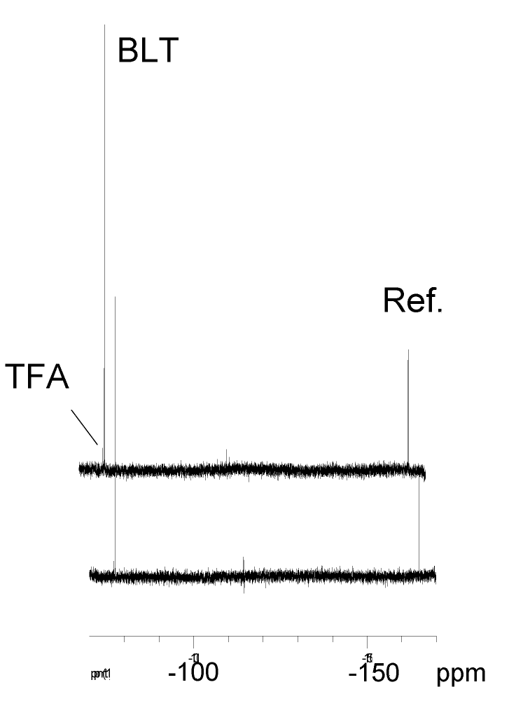

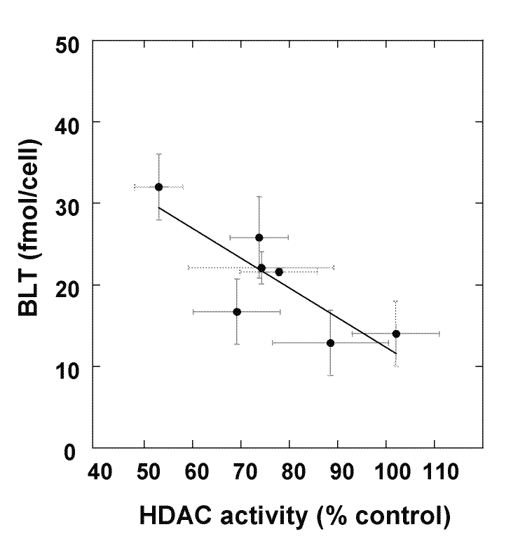

FIG. 4A illustrates the ¹⁹F MRS spectra recorded from extracts of PC3 cells. Control cells, cultured in the presence of 1 mM BLT, contained 14±4 fmol/cell BLT. This value increased significantly to 32±4 fmol/cell in cells treated with 10 μM FSAHA in the presence of BLT (P<0.0002), consistent with intracellular uncleaved BLT levels being higher when HDAC is inhibited. No signal could be detected from FSAHA, which was expected to resonate at −121 ppm. In addition, no ¹⁹F signal was observed from the lipid phase or the protein pellet of the cells.

Interestingly, TFA levels observed in both treated and control cells remained, within experimental error, unchanged. The average TFA concentration observed in control cells was 1.1±0.6 fmol/cell versus 1±1 fmol/cell in cells treated with 10 μM FSAHA. It was therefore speculated that TFA produced inside the cell was removed into the extracellular compartment. To test this hypothesis GC-MS was used to determine the levels of TFA present in cellular growth medium. In control cells the level of TFA was 5±0.4 μg/ml but was only 3.4±0.7 μg/ml in the medium obtained from 10 μM FSAHA-treated cells (P<0.03).

To confirm that the intracellular BLT levels detected by MRS are indicative of inhibition of cellular HDAC, HDAC activity and cellular BLT levels in cells treated with a range of FSAHA concentrations from 2 to 10 μM were moniotored. HDAC activity dropped significantly for all concentrations greater than 2 μM. An increase in BLT levels was observed for all FSAHA concentrations investigated and reached statistical significance when

HDAC activity dropped to 74% relative to controls. Furthermore, a negative correlation was observed between HDAC activity and the level of intracellular BLT (Rho=−0.75, P<0.05) (FIG. 4B).

³¹P MRS Can be Used to Assess HDAC Inhibition in Cells.

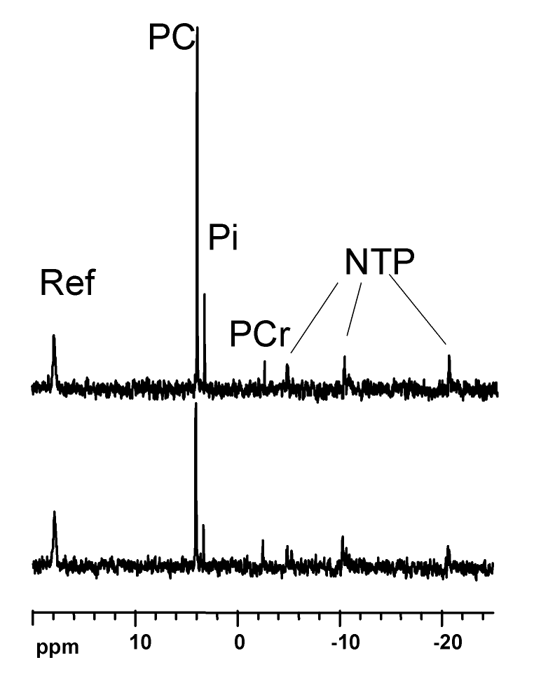

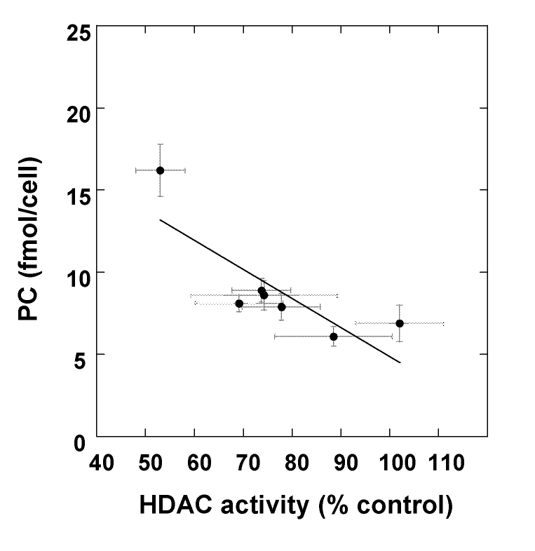

Because HDAC inhibition leads to modulation of several genes that are associated with MRS detectable metabolic changes, we questioned whether response to treatment with HDACIs is also detectable in the ³¹P MR spectrum of treated cells. ³¹P MRS was used to investigate the same PC3 samples investigated by ¹⁹F MRS. As illustrated in FIG. 5A treatment with 10 μM FSAHA resulted in a significant increase in PC levels from 7±1 fmol/cell to 16±2 fmol/cell (P<0.01). None of the other metabolites observed in the ³¹P MRS spectrum were altered. As in the case of BLT, PC levels for cells treated with lower concentrations of FSAHA also showed an increase relative to controls reaching statistical significance when HDAC activity dropped to 74% relative to controls. PC levels also negatively correlated with HDAC activity (Rho=−0.86 P<0.02) (FIG. 5B).

³¹P MRS Changes are Consistent With Depletion of Hsp90 Client Proteins cdk4 and c-Raf-1.

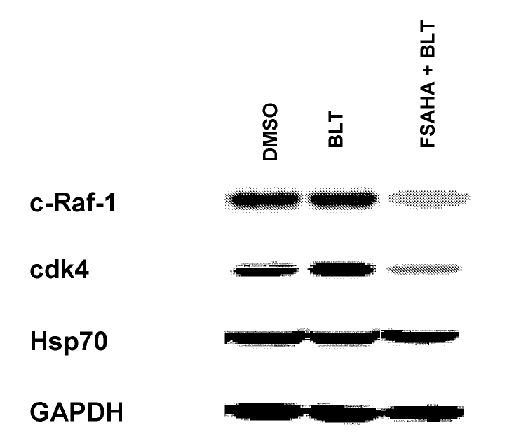

The increase in PC observed in the spectra following response to HDACI-treatment is an unusual observation and only previously reported following treatment with the Hsp90 inhibitor 17AAG (Chung Y L, Troy H, Banerji U, et al. Magnetic resonance spectroscopic pharmacodynamic markers of the heat shock protein 90 inhibitor 17-allylamino, 17-demethoxygeldanamycin (17AAG) in human colon cancer models. J Natl Cancer Inst 2003 November 5;95(21):1624-33.). Hsp90 inhibition has also been reported following HDACI treatment. To test the hypothesis that the ³¹P MRS changes observed here were associated with inhibition of Hsp90 we monitored the levels of the Hsp90 client proteins c-Raf-1 and cdk4. FIG. 6 indicates depletion of these two Hsp90 client proteins following treatment with FSAHA. However induction of Hsp70, which has also been reported following inhibition of 17AAG, was not observed in our cells (FIG. 6).

In silico modeling of the BLT-HDAC interaction and in vitro MRS studies of BLT cleavage by HDAC confirmed BLT as an HDAC substrate. BLT did not affect cell viability or HDAC activity in PC3 prostate cancer cells PC3 cells were treated, in the presence of BLT, with the HDAC inhibitor p-fluoro-suberoylanilide hydroxamic acid (FSAHA) over the range of 0 to 10 μM and HDAC activity and MRS spectra monitored. Following FSAHA treatment HDAC activity dropped, reaching 53% of control at 10 μM FSAHA. In parallel a steady increase in intracellular BLT from 14 fmol/cell to 32 fmol/cell was observed. BLT levels negatively correlated with HDAC activity consistent with higher levels of uncleaved BLT in cells with inhibited HDAC. Phosphocholine, detected by ³¹P MRS, increased from 7 fmol/cell to 16 fmol/cell following treatment with FSAHA and also negatively correlated with HDAC activity. Increased PC is probably due to HSP90 inhibition as indicated by depletion of client proteins. In summary ¹⁹F MRS of BLT, combined with ³¹P MRS, can be used to monitor HDAC activity in cells. In principle, this could be applied in vivo to noninvasively monitor HDAC.

MRS is a noninvasive method that can be readily translated to the clinic. Our investigations therefore concentrated on a derivative of the clinically relevant HDACI SAHA. We chose to concentrate our studies on FSAHA, rather than SAHA, because we reasoned that if a significant level of FSAHA accumulates intracellularly it would be possible to simultaneously monitor both the delivery of FSAHA and its effect on HDAC activity by ¹⁹F MRS. FSAHA could not be detected in any of our spectra. Therefore we conclude that the intracellular level of FSAHA is below MRS detection level (ca. 0.1 fmol/cell). Current phase I trials (Kelly W K, O'Connor O A, Krug L M, et al. Phase I study of an oral histone deacetylase inhibitor, suberoylanilide hydroxamic acid, in patients with advanced cancer. J Clin Oncol 2005 June 10;23(17):3923-31) found that the mean plasma concentration of SAHA in vivo was less than 600 mg/ml which is also expected to be below detection level.

The inhibitory effect of FSAHA was slightly lower at 10 μM than that of SAHA. We have not investigated the reasons for this difference. Nonetheless, this observation does not affect the value of our MRS findings. Since we are able to detect the effects of the less potent fluorinated inhibitor, we believe we would also be able to detect the effects of the parent inhibitor SAHA or other HDACIs of equal or greater efficacy.

Surprisingly, TFA—the cleavage product of BLT—was constant in the intracellular compartment of control and HDACI-treated cells. However an analysis of the extracellular medium demonstrated that TFA was lower by an average 1.6 μg/ml in the medium of 10 μM FSAHA-treated cells compared to controls. On average, intracellular BLT increased by 18 fmol/cell in those cells. Assuming that any TFA produced through cleavage of BLT by HDACs is removed into the extracellular medium, this would lead to TFA levels lower by 1.3 μg/ml in the medium of HDAC-inhibited cells. This number is consistent with the TFA levels determined experimentally in our extracellular medium. We conclude that TFA produced by cleavage of BLT is removed from the intracellular compartment into the medium, in line with previous findings. In vivo, depending on the rate of TFA clearance, it is possible that both BLT and TFA will present in the tumor region. However, due to the small chemical shift difference between BLT and TFA, monitoring TFA is expected to be difficult.

In addition to directly assessing HDAC activity by ¹⁹F MRS of BLT, a unique metabolic biomarker of response was afforded by using ³¹P MRS to monitor the intrinsic cellular metabolites. Inhibition of cell growth following chemotherapeutic treatment as well as signaling inhibition is typically associated with an MR visible drop in PC levels. The increase in PC observed here is therefore unusual and has previously been observed only following response to treatment with 17AAG. 17AAG causes inhibition of Hsp90, which results in depletion of its client proteins including cdk4 and c-Raf-1 as well as upregulation of Hsp70. Interestingly, HDACI treatment results in increased Hsp90 acetylation also leading to inhibition of its activity and depletion of client proteins. In the specific case of SAHA, a drop in both cdk4 and c-Raf-1 has been observed in some cases but not in others. Our Western blot analysis indicates depletion of both cdk4 and c-Raf-1 in treated cells. However we did not observe any upregulation of Hsp70. Thus it is not entirely clear if the depletion of cdk4 and c-Raf-1 is a direct result of HDAC inhibition or occurs subsequent to Hsp90 acetylation following HDAC inhibition. Nonetheless, we believe that the increase in PC observed by MRS is associated with the depletion of cdk4 and c-Raf-1. The mechanism linking cdk4 and c-Raf-1 with modulation of PC remains to be elucidated, but the results described here following HDACI-treatment are entirely consistent with our earlier observations. It should be noted that we had previously also observed an increase in GPC following response to 17AAG. In this study, this metabolite remained below detection level and thus it is not clear if its levels are altered by HDACI-treatment.

Experiments indicate that an intraperitoneal injection of 100 mg/kg of BLT once a week on three subsequent weeks (a schedule consistent with monitoring response to HDACIs in vivo) results in no detectable toxicity to the animal. Importantly, this dose was sufficient to produce an MRS visible BLT signal in subcutaneous PC3 tumors with a temporal resolution of 5 minutes at 4.7 T. The BLT signal remained detectable within the tumor region for over 2 hours. As expected, the TFA peak could not be easily resolved in our preliminary in vivo studies. Intratumoral BLT levels may be higher in HDACI-treated tumors compared to controls, and therefore tumoral BLT levels may be used to assess HDAC activity in vivo. Downstream metabolic biomarkers also may be assessed in our preliminary studies. We were able to acquire a ³¹P spectrum from PC3 subcutaneous tumors in 30 minutes providing a means for monitoring PC levels. This is consistent with previous studies in which an increase in PC could be monitored as an indicator of response to 17AAG treatment. ¹H MRS, with its greater sensitivity compared to ³¹P, could also be used to monitor the total choline signal as a downstream metabolic marker of response to HDAC inhibition.

In summary, the compositions and methods of the present disclosure provide means for noninvasively monitoring response to HDACIs. ¹⁹F MRS of the targeted molecular imaging agent BLT can be used to monitor delivery and activity of HDACIs at the tumor site, while ³¹P MRS can be used to monitor the downstream metabolic consequences of HDAC inhibition. Together, these two MRS methods provide both a direct marker of HDAC inhibition and a downstream biomarker of cellular response to the inhibition. Combining both indicators provides a more powerful tool than a single marker alone, particularly at lower levels of HDAC inhibition when the changes observed in either marker alone are relatively small. The combination of ¹⁹F and ³¹P (or ¹H) MRS could thus serve as a reliable noninvasive modality to assess HDAC inhibition.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

Therefore, the present disclosure is well adapted to attain the ends and advantages mentioned as well as those that are inherent therein. While numerous changes may be made by those skilled in the art, such changes are encompassed within the spirit of this disclosure as illustrated, in part, by the appended claims.

### REFERENCES

1. Marks P, Rifkind R A, Richon V M, Breslow R, Miller T, Kelly W K. Histone deacetylases and cancer: causes and therapies. Nat Rev Cancer 2001 December;1(3):194-202.

2. Drummond D C, Noble C O, Kirpotin D B, Guo Z, Scott G K, Benz C C. Clinical development of histone deacetylase inhibitors as anticancer agents. Annu Rev Pharmacol Toxicol 2005;45:495-528.

3. Marks P A, Richon V M, Miller T, Kelly W K. Histone deacetylase inhibitors. Adv Cancer Res 2004;91 137-68.

4. Butler L M, Agus D B, Scher H I, et al. Suberoylanilide hydroxamic acid, an inhibitor of histone deacetylase, suppresses the growth of prostate cancer cells in vitro and in vivo. Cancer Res 2000 September 15;60(18):5165-70.

5. Kuefer R, Hofer M D, Altug V, et al. Sodium butyrate and tributyrin induce in vivo growth inhibition and apoptosis in human prostate cancer. Br J Cancer 2004 January 26;90(2):535-41.

6. Fronsdal K, Saatcioglu F. Histone deacetylase inhibitors differentially mediate apoptosis in prostate cancer cells. Prostate 2005 February 15;62(3):299-306.

7. Kelly W K, Richon V M, O'Connor O, et al. Phase I clinical trial of histone deacetylase inhibitor: suberoylanilide hydroxamic acid administered intravenously. Clin Cancer Res 2003 September 1;9(10 Pt 1):3578-88.

8. Sandor V, Bakke S, Robey R W, et al. Phase I trial of the histone deacetylase inhibitor, depsipeptide (FR901228, NSC 630176), in patients with refractory neoplasms. Clin Cancer Res 2002 March;8(3):718-28.

9. Chavez-Blanco A, Segura-Pacheco B, Perez-Cardenas E, et al. Histone acetylation and histone deacetylase activity of magnesium valproate in tumor and peripheral blood of patients with cervical cancer. A phase I study. Mol Cancer 2005 July 7;4(1):22.

10. Kelly W K, O'Connor O A, Krug L M, et al. Phase I study of an oral histone deacetylase inhibitor, suberoylanilide hydroxamic acid, in patients with advanced cancer. J Clin Oncol 2005 June 10;23(17):3923-31.

11. Vigushin D M, Coombes R C. Histone deacetylase inhibitors in cancer treatment. Anticancer Drugs 2002 January;13(1):1-13.

12. Villar-Garea A, Esteller M. Histone deacetylase inhibitors: understanding a new wave of anticancer agents. Int J Cancer 2004 November 1;112(2):171-8.

13. Archer S Y, Meng S, Shei A, Hodin R A. p21(WAF1) is required for butyrate-mediated growth inhibition of human colon cancer cells. Proc Natl Acad Sci USA 1998 June 9;95(12):6791-6.

14. Blagosklonny M V, Robey R, Sackett D L, et al. Histone deacetylase inhibitors all induce p21 but differentially cause tubulin acetylation, mitotic arrest, and cytotoxicity. Mol Cancer Ther 2002 September;1(11):937-41.

15. Glaser K B, Staver M J, Waring J F, Stender J, Ulrich R G, Davidsen S K. Gene expression profiling of multiple histone deacetylase (HDAC) inhibitors: defining a common gene set produced by HDAC inhibition in T24 and MDA carcinoma cell lines. Mol Cancer Ther 2003 February;2(2):151-63.

16. Mitsiades C S, Mitsiades N S, McMullan C J, et al. Transcriptional signature of histone deacetylase inhibition in multiple myeloma: biological and clinical implications. Proc Natl Acad Sci USA 2004 January 13;101(2).540-5.

17. Richon V M, Sandhoff T W, Rifkind R A, Marks P A. Histone deacetylase inhibitor selectively induces p21WAF1 expression and gene-associated histone acetylation. Proc Natl Acad Sci USA 2000 August 29;97(18):10014-9.

18. Almenara J, Rosato R, Grant S. Synergistic induction of mitochondrial damage and apoptosis in human leukemia cells by flavopiridol and the histone deacetylase inhibitor suberoylanilide hydroxamic acid (SAHA). Leukemia 2002 July;16(7):1331-43.

19. Kim M S, Kwon H J, Lee Y M, et al. Histone deacetylases induce angiogenesis by negative regulation of tumor suppressor genes. Nat Med 2001 April;7(4):437-43.

20. Park S H, Lee S R, Kim B C, et al. Transcriptional regulation of the transforming growth factor beta type II receptor gene by histone acetyltransferase and deacetylase is mediated by NF-Y in human breast cancer cells. J Biol Chem 2002 February 15;277(7):5168-74.

21. Scott G K, Marden C, Xu F, Kirk L, Benz C C. Transcriptional repression of ErbB2 by histone deacetylase inhibitors detected by a genomically integrated ErbB2 promoter-reporting cell screen. Mol Cancer Ther 2002 April;1(6):385-92.

22. McLaughlin F, La Thangue N B. Histone deacetylase inhibitors open new doors in cancer therapy. Biochem Pharmacol 2004 September 15;68(6):1139-44.

23. Gelmon K A, Eisenhauer E A, Harris A L, Ratain M J, Workman P. Anticancer agents targeting signaling molecules and cancer cell environment: challenges for drug development? J Natl Cancer Inst 1999 August 4;91(15):1281-7.

24. Parulekar W R, Eisenhauer E A. Phase I trial design for solid tumor studies of targeted, non-cytotoxic agents: theory and practice. J Natl Cancer Inst 2004 July 7;96(13):990-7.

25. Griffiths J R, Glickson J D. Monitoring pharmacokinetics of anticancer drugs: non-invasive investigation using magnetic resonance spectroscopy. Adv Drug Deliv Rev 2000 March 15;41(1):75-89.

26. Klomp D W, Van Laarhoven H W, Kentgens A P, Heerschap A. Optimization of localized 19F magnetic resonance spectroscopy for the detection of fluorinated drugs in the human liver. Magn Reson Med 2003 August;50(2):303-8.

27. Wolf W, Presant C A, Waluch V 19F-MRS studies of fluorinated drugs in humans. Adv Drug Deliv Rev 2000 March 15;41(1):55-74.

28. Yu J X, Kodibagkar V D, Cui W, Mason R P, 19F: a versatile reporter for non-invasive physiology and pharmacology using magnetic resonance. Curr Med Chem 2005; 12(7): 819-48.

29. Seddon B M, Maxwell R J, Honess D J, et al. Validation of the fluorinated 2-nitroimidazole SR-4554 as a noninvasive hypoxia marker detected by magnetic resonance spectroscopy. Clin Cancer Res 2002 July;8(7):2323-35.

30. Stegman L D, Rehemtulla A, Beattie B, et al. Noninvasive quantitation of cytosine deaminase transgene expression in human tumor xenografts with in vivo magnetic resonance spectroscopy. Proc Natl Acad Sci USA 1999 August 17;96(17):9821-6.

31. Negendank W. Studies of human tumors by MRS: a review. NMR Biomed 1992 September-October;5(5):303-24.

32. de Certaines J D, Larsen V A, Podo F, Carpinelli G, Briot O, Henriksen O. In vivo 31P MRS of experimental tumours. NMR Biomed 1993 November-December;6(6):345-65.

33. Evelhoch J L, Gillies R J, Karczmar G S, et al. Applications of magnetic resonance in model systems: cancer therapeutics. Neoplasia 2000 January-April;2(1-2) 152-65.

34. Beloueche-Babari M, Jackson L E, Al-Saffar N M S, Workman P, Leach M O, Ronen S M. Magnetic resonance spectroscopy monitoring of MAPK signaling inhibition Cancer Res 2005; 65(8):3356-63.

35. Ronen S M, Jackson L E, Beloueche M, Leach M O. Magnetic resonance detects changes in phosphocholine associated with Ras activation and inhibition in NIH 3T3 cells. Br J Cancer 2001 March 2;84(5):691-6.

36. Beloueche-Babari M, Jackson L, Salman-Al Saffar N, et al. Identification of Magnetic Resonance Detectable Metabolic Changes Associated with Inhibition of Phosphoinositide 3-Kinase Signalling in Human Breast Cancer Cells. Mol Cancer Therp 2005; in press.

37. Chung Y L, Troy H, Banerji U, et al. Magnetic resonance spectroscopic pharmacodynamic markers of the heat shock protein 90 inhibitor 17-allylamino, 17-demethoxygeldanamycin (17AAG) in human colon cancer models. J Natl Cancer Inst 2003 November 5;95(21):1624-33.

38. Beloueche M, Payne, G. S., Banerji, U., Collins. D. J., Dzik-Jurasz, A. S. K., Workman, P., Judson. I. R., Leach, M. O., Ronen, S. M. MRS monitoring of the response to 17AAG treatment: from the laboratory to the clinic. Proceedings of 20th ESMRMB; 2003; Rotterdam; 2003.

39. Yu X, Guo Z S, Marcu M G, et al. Modulation of p53, ErbB1, ErbB2, and Raf-1 expression in lung cancer cells by depsipeptide FR901228. J Natl Cancer Inst 2002 April 3;94(7):504-13.

40. Podo F Tumour phospholipid metabolism. NMR Biomed 1999 November;12(7):413-39.

41. Mori N D, R. Natarajan, K. Singh, K. K., Bhujwalla, Z. M. Loss of p53 function in colon cancer cells results in increased phosphocholine and total choline. Proceedings of 10th ISMRM; 2002; Hawai, 2002. p. 28.

42. Fuino L, Bali P, Wittmann S, et al. Histone deacetylase inhibitor LAQ824 down-regulates Her-2 and sensitizes human breast cancer cells to trastuzumab, taxotere, gemcitabine, and epothilone B. Mol Cancer Ther 2003 October;2(10):971-84.

43. Bali P, Pranpat M, Bradner J, et al. Inhibiition of histone deacetylase 6 acetylates and disrupts the chaperone function of heat shock protein 90: A novel basis of antileukemia activity of histone deacetylase inhibitors. J Biol Chem 2005 June 2;280(29):26729-34.

44. Berman H M, Westbrook J, Feng Z, et al. The Protein Data Bank. Nucleic Acids Res 2000 January 1;28(1):235-42.

45. Clark R D, Strizhev A, Leonard J M, Blake J F, Matthew J B. Consensus scoring for ligand/protein interactions. J Mol Graph Model 2002 January;20(4):281-95.

46. Stowell J C, Huot R I, Van Voast L. The synthesis of N-hydroxy-N′-phenyloctanediamide and its inhibitory effect on proliferation of AXC rat prostate cancer cells. J Med Chem 1995 April 14;38(8):1411-3.

47. Tyagi R K, Azrad A, Degani H, Salomon Y Simultaneous extraction of cellular lipids and water-soluble metabolites: evaluation by NMR spectroscopy. Magn Reson Med 1996 February;35(2):194-200.

48. Scott B F, Mactavish D, Spencer C, Strachan W M J, Muir DCG. Haloacetic acids in Canadian lake waters and precipitation. Environmental Science & Technology 2000;34:4266-72.

49. Somoza J R, Skene R J, Katz B A, et al. Structural snapshots of human HDAC8 provide insights into the class I histone deacetylases. Structure (Camb) 2004 July;12(7):1325-34.

50. Hanahan D, Weinberg R A. The hallmarks of cancer. Cell 2000 January 7;100(1):57-70.

51. Sawyers C. Targeted cancer therapy. Nature 2004 November 18;432(7015):294-7.

52. Wolffe A P. Chromatin remodeling: why it is important in cancer. Oncogene 2001 May 28;20(24):2988-90.

53. Cress W D, Seto E. Histone deacetylases, transcriptional control, and cancer. J Cell Physiol 2000 July;184(1):1-16.

54. Di Gennaro E, Bruzzese F, Caraglia M, Abruzzese A, Budillon A. Acetylation of proteins as novel target for antitumor therapy; review article. Amino Acids 2004 July;26(4):435-41.

55. Xu A S, Kuchel P W. Characterisation of erythrocyte transmembrane exchange of trifluoroacetate using 19F-NMR: evidence for transport via the monocarboxylate transporter. Biochim Biophys Acta 1993 July 25;1150(1):35-44.

56. Leach M O, Verrill M, Glaholm J, et al. Measurements of human breast cancer using magnetic resonance spectroscopy: a review of clinical measurements and a report of localized 31P measurements of response to treatment. NMR Biomed 1998 November;11(7):314-40.

57. Workman P Combinatorial attack on multistep oncogenesis by inhibiting the Hsp90 molecular chaperone. Cancer Lett 2004 April 8;206(2):149-57.

58. Neckers L. Development of small molecule Hsp90 inhibitors: utilizing both forward and reverse chemical genomics for drug identification. Curr Med Chem 2003 May;10(9):733-9.

59. Kovacs J J, Murphy P J, Gaillard S, et al. HDAC6 Regulates Hsp90 Acetylation and Chaperone-Dependent Activation of Glucocorticoid Receptor. Mol Cell 2005 May 27;18(5):601-7.

60. Peart M J, Smyth G K, van Laar R K, et al. Identification and functional significance of genes regulated by structurally different histone deacetylase inhibitors. Proc Natl Acad Sci USA 2005 March 8;102(10):3697-702.

61. Stowell J., Huot R., and VanVoast L., J. Med. Chem. 38: 1411 (1995).

62. Khawli L. A., Kassis A. I., Nucl. Med. Biol. 19: 297 (1992).

What is claimed is:

1. A histone deacetylase substrate comprising a compound of the following formula (I):

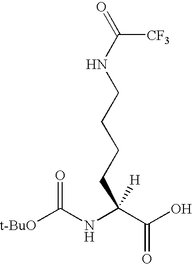

2. A composition formed from the activity of histone deacetylase on the deacetylase substrate of claim 1.

3. A histone deacetylase inhibitor comprising a compound represented by formula (II): 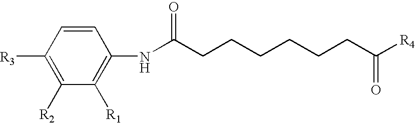
wherein R₁, R₂, and R₃ individually represent an F or H, and R₄ represents NHOH or OEt.

4. The histone deacetylase inhibitor of claim 3 wherein R₁ represents F, R₂ represents H, R₃ represents H, and R₄ represents NHOH.

5. The histone deacetylase inhibitor of claim 3 wherein R₁ represents H, R₂ represents F, R₃ represents H, and R₄ represents NHOH.

6. The histone deacetylase inhibitor of claim 3 wherein R₁ represents H, R₂ represents H, R₃ represents F, and R₄ represents NHOH.

7. The histone deacetylase inhibitor of claim 3 wherein R₁ represents F, R₂ represents H, R₃ represents H, and R₄ represents OEt.

8. The histone deacetylase inhibitor of claim 3 wherein R₁ represents H, R₂ represents F, R₃ represents H, and R₄ represents OEt.

9. The histone deacetylase inhibitor of claim 3 wherein R₁ represents H, R₂ represents H, R₃ represents F, and R₄ represents OEt.

10. A histone deacetylase inhibitor comprising a compound represented by formula (III):

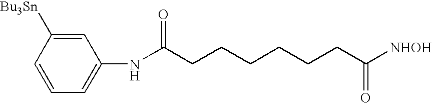

11. A histone deacetylase inhibitor comprising a compound represented by formula (IV):

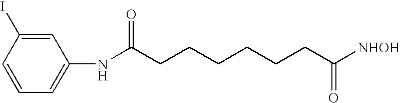

12. A method for detecting histone deacetylase activity comprising introducing a compound according to claim 1 to a plurality of cells and monitoring the cells with magnetic resonance spectroscopy.

13. The method of claim 12 wherein the cells are monitored for cleavage of the compound according to claim 1.

14. The method of claim 12 further comprising introducing a histone deacetylase inhibitor to the cells.

15. The method of claim 12 wherein the cells are monitored for a change in a biomarker chosen from at least one of a cellular metabolite, phosphocholine, and glycerophosphocholine.

16. The method of claim 12 wherein the magnetic resonacne spectroscopy is chosen from at least one of ¹⁹F magnetic resonacne spectroscopy, ³¹P magnetic resonacne spectroscopy, and ¹H magnetic resonacne spectroscopy.

17. The method of claim 12 further comprising introducing a therapeutic agent or a histone deacetylase inhibitor or both to the cells before monitoring the cells with magnetic resonance spectroscopy.

18. The method of claim 17 wherein the therapeutic agent is a histone deacetylase inhibitor.
